(19) United States
(12) Patent Application Publication
(10) Pub. No.: US 2026/0344982 A1
(43) Pub. Date: Nov. 5, 2026

(54) MEDICAL DEVICES, SYSTEMS, AND RELATED METHODS FOR POSITIONING AND DELIVERING SUTURES

(71) Applicant: Boston Scientific Scimed, Inc., Maple Grove, MN (US)

(72) Inventors: John Mims, Austin, TX (US); Jason N. Mehta, Austin, TX (US); Thomas Neudeck, Austin, TX (US); Greg T Prevatt, Austin, TX (US); Luis Amador, Pflugerville, TX (US)

(73) Assignee: Boston Scientific Scimed, Inc., Maple Grove, MN (US)

(21) Appl. No.: 19/660,616

(22) Filed: Apr. 28, 2026

Related U.S. Application Data
(60) Provisional application No. 63/797,413, filed on Apr. 30, 2025.

(51) Int. Cl. A61B 1/00; A61B 1/018; A61B 1/05; A61B 1/06; A61B 17/04

(52) U.S. Cl. CPC A61B 1/00137; A61B 1/00066; A61B 1/00087; A61B 1/00148; A61B 1/018; A61B 1/05; A61B 1/0676; A61B 17/0469

(57) ABSTRACT

The disclosure includes a medical system including: a handle, a shaft, and a suture end cap assembly. The shaft may extend from a proximal end at a distal end of the handle to a distal end. The shaft may include one or more channels extending to an opening at the distal end of the shaft. The one or more channels may include a first working channel. The suture end cap assembly may extend distally from the distal end of the shaft. The suture end cap assembly may include a needle, a needle guard, and a first working channel opening corresponding to the first working channel. A common axis may intersect a longitudinal axis of the suture end cap assembly and a center point of the first working channel opening. A shaft of an end effector assembly of the medical system may be slidably received within the first working channel.

### CROSS-REFERENCE TO RELATED APPLICATION(S)

This application claims the benefit of priority to U.S. Provisional Application No. 63/797,413, filed on April 30, 2025, which is incorporated by reference herein in its entirety.

### TECHNICAL FIELD

This disclosure relates generally to medical devices, systems, and related methods. More particularly, this disclosure relates to needles for use in suturing tissue, medical devices for aligning tissue with a suture needle, and methods for joining tissue, among other aspects.

### BACKGROUND

In some medical procedures, such as endoscopic sleeve gastroplasty (ESG), it may be necessary or useful to fixedly connect a portion of tissue to another portion of tissue. Attaching portions of tissue, such as to hold together a wound or damaged tissue, with one or more sutures may allow adhesions to form between the two tissues so that the attachment remains after the sutures are absorbed or removed. Tissue attachment may involve piercing tissue with the needle and manipulating the needle to pull or thread a suture attached to the needle through the tissue. In some procedures, a needle assembly, including a needle and suture, may be positioned at a distal end of a shaft or insertion portion of a medical device. The shaft or insertion portion (e.g., with the needle assembly coupled to the distal end) may be inserted through a natural or artificial orifice (e.g., an esophagus) of the patient and navigated through tortuous bodily lumens or cavities of the patient to the target site. The user may actuate the medical device to deliver sutures through tissue(s) at the target site. However, consistent delivery of sutures through tissues with appropriate penetration, length, or position relative to the target site and other sutures may be difficult, time-consuming, or otherwise negatively affect the treatment.

### SUMMARY

The disclosed devices, systems, and methods may, among other benefits, help a user in positioning and delivering suture at a target site. Each of the aspects disclosed herein may include one or more features described in connection with any of the other disclosed aspects.

The disclosure includes a medical system. For example, the disclosed medical system may include a handle, a shaft, and a suture end cap assembly. The shaft may extend from a proximal end of the shaft at a distal end of the handle to a distal end of the shaft. The shaft may include one or more channels extending from the proximal end of the shaft to a distal opening at the distal end of the shaft. The one or more channels may include a first working channel. The suture end cap assembly may extend distally from the distal end of the shaft. The suture end cap assembly may include a needle, a needle guard, and a first working channel opening corresponding to the first working channel. A common axis may intersect a longitudinal axis of the suture end cap assembly and a center point of the first working channel opening. An end effector of the system may be slidably received within the first working channel.

The medical system may include one or more of the following features. For example, the common axis may be a first common axis. A second common axis, perpendicular to the first common axis, may include the center point of the first working channel opening. Alternatively, the center point of the first working channel opening may be positioned opposite to the needle guard relative to a second common axis that is perpendicular to the first common axis. The one or more channels may include a second working channel. The suture end cap assembly may include a second working channel opening corresponding to the second working channel. The common axis (e.g., first common axis) may intersect a center point of the second working channel opening. The system may further include a control member. The one or more channels of the shaft may include a control member channel in which the control member is slidably positioned therein. The common axis (e.g., first common axis) may intersect a center point of the control member channel. The control member may be actuatable to control a position of the needle. The handle may include a proximal portion and a distal portion. The distal portion of the handle may be rotatable relative to the proximal portion of the handle. The shaft may be coupled to and rotatable with the distal portion of the handle. The end effector may be movable relative to the suture end cap assembly to control a length of a suture delivered by the suture end cap assembly to tissue. The suture end cap assembly may be fixedly coupled to the distal end of the shaft. The end effector may be integrated with the handle. The suture end cap assembly may include one or more lighting elements and one or more imaging elements. A central longitudinal axis of the end effector may be coaxial with a central longitudinal axis of the shaft.

Aspects of the disclosure include a medical device. The medical device may include a handle and a shaft extending from a proximal end of the shaft a distal end of the handle to a distal end of the shaft. The shaft may include a first working channel and a second working channel. Each of the first working channel and the second working channel may extend from the proximal end of the shaft to a distal opening at the distal end of the shaft. The medical device includes a suture end cap assembly at the distal end of the shaft. The suture end cap assembly may include a needle driver, a needle coupled to the needle driver, and a needle guard. A common plane may extend along a central longitudinal axis of the shaft. The common plane may bisect the needle and a center point of the first working channel opening.

The medical device may include one or more of the following features. For example, the needle driver may be actuatable to move and intersect an axis perpendicular to the central longitudinal axis. The medical device may further comprise an end effector assembly integrated with the handle. The end effector assembly may include a shaft and an end effector at the distal end of the shaft. A portion of the end effector assembly may be positioned within the first working channel. The end effector may include a corkscrew or helical shape.

Various other aspects of this disclosure include a medical device. The medical device may include a handle and a shaft extending from a proximal end of the shaft a distal end of the handle to a distal end of the shaft. The medical device further includes a suture end cap assembly at the distal end of the shaft. The suture end cap assembly may include a suture assembly including a distal face, a needle, and a needle guard. The needle guard may include a longitudinal axis and may be connected to the distal face. The suture end cap assembly may include a first distal opening and second distal opening, each distal opening may be in communication with one or more lumens of the shaft and may include a central axis. A common axis may intersect the central axis of the first distal opening, the central axis of the second distal opening, and the longitudinal axis of the needle guard.

The foregoing general description of the illustrative embodiments and the following detailed description thereof are merely exemplary aspects of the teachings of this disclosure and are not restrictive.

### BRIEF DESCRIPTION OF FIGURES

The accompanying drawings are incorporated in and constitute a part of this specification. These drawings illustrate aspects of the disclosure that, together with the written descriptions herein, serve to explain this disclosure. Each drawing depicts one or more exemplary aspects according to this disclosure, as follows:

FIG. 1A depicts a side view of an exemplary suture delivery system, including an insertion device and a suture delivery assembly, including an enlarged view of a distal portion of the suture delivery system.

FIG. 1B depicts a cross-sectional view of a shaft of the insertion device.

FIG. 2A depicts a proximally facing view of a distal end of the shaft, and the suture delivery assembly.

FIG. 2B depicts a longitudinal cross-sectional view of the distal end of the shaft and the suture delivery assembly.

FIG. 3A depicts a distal end of the suture delivery system engaged with tissue at the target site, with the tissue in a first position relative to the suture delivery system, and FIG. 3B depicts a suture delivered to the tissue at the target site having a first length.

FIG. 4A depicts the distal end of the suture delivery system engaged with tissue at the target site, with the tissue in a second position relative to the suture delivery system, and FIG. 4B depicts a suture delivered to the tissue at the target site having a second length.

FIG. 5A depicts the distal end of the suture delivery system engaged with tissue at the target site, with the tissue in a third position relative to the suture delivery system, and FIG. 5B depicts a suture delivered to the tissue at the target site having a third length.

### DETAILED DESCRIPTION

Reference will now be made in detail to examples of the present disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device. When used herein, “proximal” refers to a position relatively closer to the exterior of the body or closer to a medical professional using the medical device. In contrast, “distal” refers to a position relatively further away from the medical professional using the medical device, or closer to the interior of the body. As used herein, the terms “comprises,” “comprising,” “has,” “having,” “includes,” “including,” or other variations thereof, are intended to cover a non-exclusive inclusion, such that a device or method that comprises a list of elements does not include only those elements, but may include other elements not expressly listed or inherent thereto. Further, as used herein, the term “or” indicates non-exclusive inclusion, such that a list of elements includes one or more, or all, of the listed elements unless otherwise indicated. The term “or” is used disjunctively, such that “at least one of A or B” includes, (A), (B), (A and A), (A and B), etc. For example, the term “or” also includes “and/or.” Unless stated otherwise, the term “exemplary” is used in the sense of “example” rather than “ideal.” As used herein, the terms “about,” “substantially,” and “approximately,” indicate a range of values within +/- 10% of a stated value.

The medical devices, systems, and methods depicted in the figures may be used in various minimally invasive medical procedures to access and navigate bodily lumens or cavities. These medical devices generally include an elongated sheath or shaft that can be inserted through a natural or artificial opening in a patient's body. For example, the disclosed medical devices may be extended through an esophagus of a patient to access the gastrointestinal tract. In another example, the disclosed medical devices may be extended through a rectum of the patient to access the gastrointestinal tract (e.g., a lower portion of the gastrointestinal tract). The disclosed medical devices may be used to position and deliver sutures to a tissue(s) at a target site. In some examples, a distal end of the disclosed medical devices may be positioned within a stomach of the patient near the mucosa layer of the stomach to deliver sutures through the mucosa layer and the serosa layer.

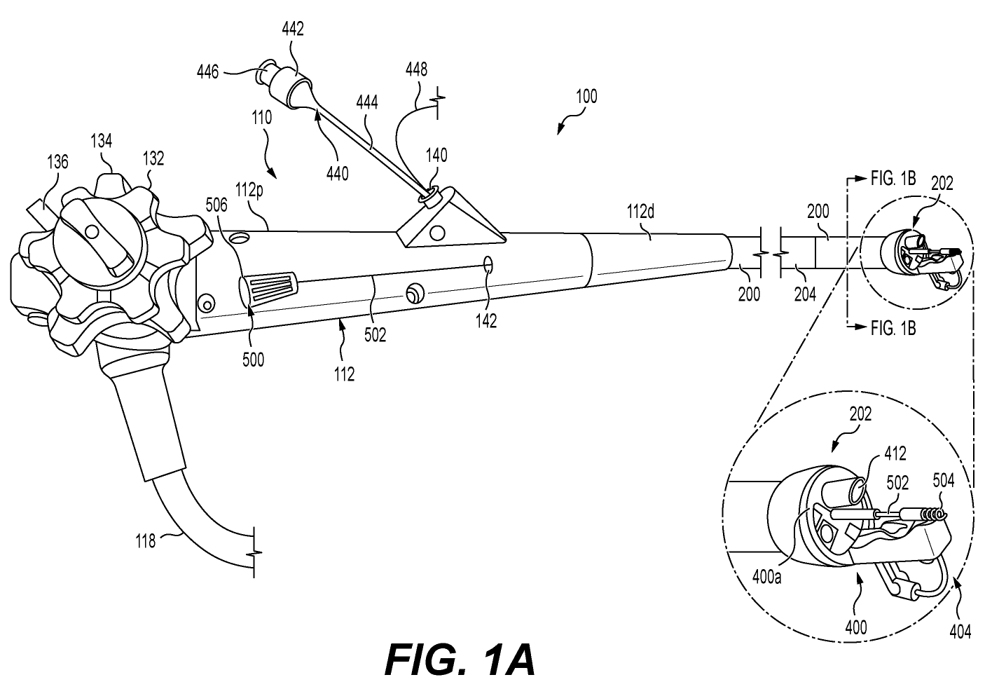

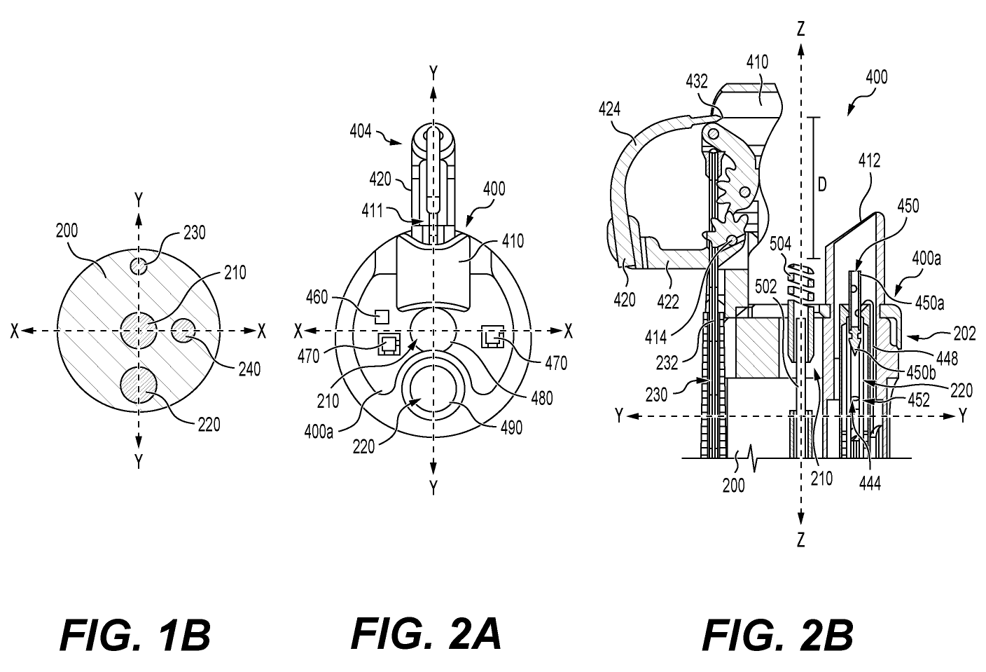

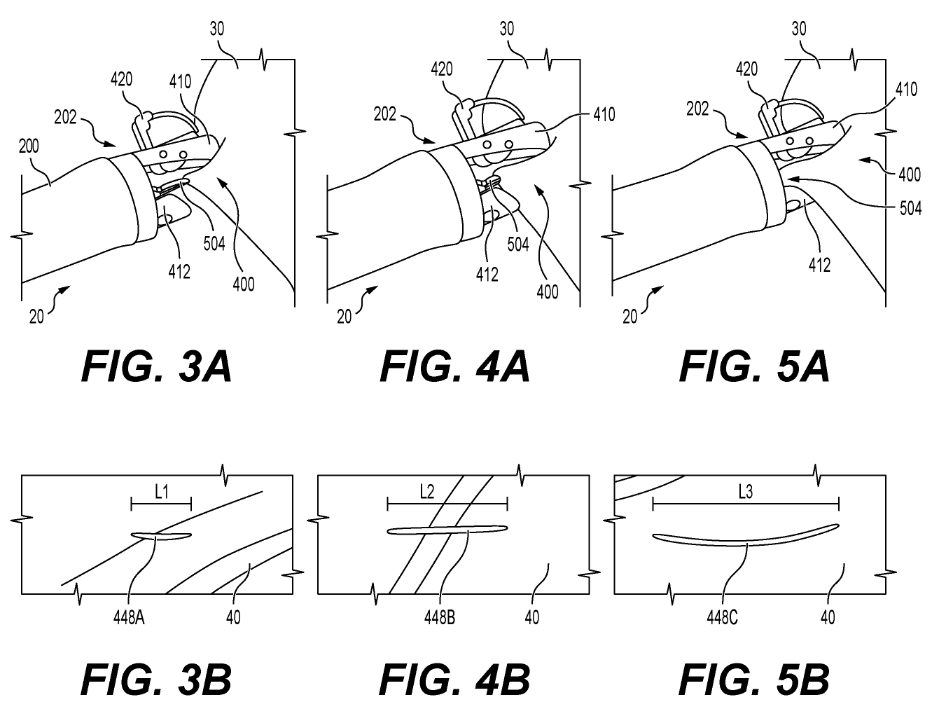

FIG. 1A depicts an exemplary insertion device, an insertion device 110, of an exemplary medical system, a medical system 100, according to aspects of the disclosure. In some aspects, insertion device 110 may be an endoscope. Although an endoscope may be referenced herein, it will be appreciated that the disclosure also encompasses duodenoscope, bronchoscopes, gastroscopes, EUS scopes, colonoscopes, ureteroscopes, bronchoscopes, laparoscopes, cystoscopes, aspiration scopes, sheaths, catheters, or similar devices. A reference to an endoscope herein should be understood to encompass any of the above medical devices. Insertion device 110 includes a handle 112 and an insertion portion, sheath, or shaft 200 extending distally from a distal end of handle 112 to a distal end 202 of shaft 200. Insertion device 110 may include a suture end cap assembly 400 fixedly attached at distal end 202 of shaft 200. As will be discussed in further detail in later sections of this disclosure, suture end cap assembly 400 includes a suture assembly 404 configured to pierce or penetrate a target (e.g., tissue 30, 40 FIGS. 3A-5B) at a target site (e.g., stomach lining 20) to install or deliver one or more sutures through the target. Furthermore, medical system 100 may include a medical instrument 500 (e.g., a helical end effector 504) coupled to or otherwise integrated into insertion device 110. As discussed in detail below, end effector 504 may be extendable within and relative to a portion of suture assembly 404, such that captured material (e.g., tissue 30, 40) is positioned appropriately relative to suture assembly 404.

Handle 112 of insertion device 110 includes a first, proximal portion 112p and a second, distal portion 112d. In some embodiments, distal portion 112d may be rotatable along a longitudinal axis of handle 112 relative to proximal portion 112p. Shaft 200 may be coupled (e.g., removably coupled or fixedly coupled) to distal portion 112d and configured to rotate with distal portion 112d. An operator may rotate shaft 200 and distal portion 112drelative to proximal portion 112p to change an orientation of suture end cap assembly 400, including suture assembly 404, relative to a target site to change an orientation that sutures are delivered to the target site. For example, in an exemplary procedure, the operator may deliver a first suture along a first axis, rotate distal portion 112d approximately 90 degrees relative to proximal portion 112p, and deliver a second suture along a second axis, the second axis being offset from the first axis by approximately 90 degrees from the first direction. In an exemplary embodiment, distal portion 112d and shaft 200 may be rotatable in a clockwise or counterclockwise direction at least 360 degrees relative to proximal portion 112p. In some embodiments, distal portion 112d and shaft 200 may be rotatable at least 180 degrees in the clockwise or counterclockwise direction relative to proximal portion 112p. As mentioned above, one or more medical devices or instruments may be integrated with insertion device 110. In an exemplary embodiment, medical system 100 may include medical instrument 500. One or more portions of medical instrument 500 may be integrally formed or integrated with handle 112 of insertion device 110. In some examples, a shaft (e.g., shaft 502) of medical instrument 500 may distally extended through a port (e.g., port 142) of insertion device 110 and coupled to insertion device 110 at the port.

Handle 112 may include one or more actuators for controlling various functions of insertion device 110 or for provision of air, water, suction, etc. Insertion device 110 may include an umbilicus 118 for purposes of connecting insertion device 110 to sources of, for example, air, water, suction, power, etc., as well as to image processing and/or viewing equipment. In some aspects, handle 112 may include a first actuator 132 and a second actuator 134 for controlling (e.g., bending or deflecting) a steerable section 204. Steerable section 204 may be, for example, an articulation joint. In an exemplary embodiment, handle 112 may include actuators 132, 134 for left, right, up, and/or down control of steerable section 204. For example, one of actuators 132, 134 may provide left/right control of steerable section 204, and the other of actuators 132, 134 may provide up/down control of steerable section 204. The operator may control a position of suture end cap assembly 400 relative to the target site via control of steerable section 204. The one or more actuators of handle 112 may take various forms such as buttons, knobs, switches, levers, dials, or other structures which are known or may become known in the art. In some embodiments, the one or more actuators may include a third actuator 136 for controlling delivery of sutures using suture end cap assembly 400 including suture assembly 404.

Handle 112 may also include one or more ports for introducing instruments or exchanging components. In some embodiments, handle 112 may include two ports, for example, a suture exchange port 140 and an end effector port 142. End effector port 142 may provide access for introducing various end effectors or medical instruments through shaft 200 to the target site. Suture exchange port 140 may allow for the insertion, removal, or exchange of a needle exchange device 440 of suture assembly 404 through shaft 200.

Now referring to FIG. 1B, which depicts a lateral cross-section of shaft 200 from the indicated position of the cross-sectional lines labeled “FIG. 1B” in FIG. 1A. Shaft 200 may include one or more lumens or channels extending from a proximal end of shaft 200 to distal end 202 of shaft 200. For example, the one or more channels may include a first working channel 210 and a second working channel 220. First working channel 210 may be in communication with end effector port 142 and may be configured to slidably receive an end effector inserted therethrough. Second working channel 220 may be in communication with suture exchange port 140 and may be configured to slidably receive at least a portion of needle exchange device 440. The one or more channels may further include control member channel 230 configured to slidably receive one or more control members therethrough. For example, control member channel 230 may include a control member 232 (FIG. 2B) operably connected to suture assembly 404 and configured to actuated one or more components of suture assembly 404. Control member 232 may be slidably received within control member channel 230. The one or more channels of shaft 200 may include an electronics channel 240 configured to house various electrical components, cable(s), wiring, etc. In some aspects, electronics channel 240 may house one or more electrical components, cables, wires, etc. for powering or communicating with one or more electrical components at distal end 202 or suture end cap assembly 400. For example, electronics channel 240 may contain electrical wires that provide power or signals to one or more lighting elements, imaging elements, or sensing elements of distal end 202 of shaft 200. It is noted that, although FIG. 1B illustrates channels 210, 220, 230, and 240 as being shaded in the cross-sectional view, that representation is indicative of components being positioned within those channels for the system 100 depicted in FIG. 1B. Furthermore, if shaft 200 were to be depicted alone, without additional components extending therethrough to form system 100, channels 210, 220, 230, and 240 would be empty and not shaded in a cross-sectional view.

Now referring to FIGS. 2A-2B, which depict distal end 202 of shaft 200 and various aspects of suture end cap assembly 400. Suture end cap assembly 400 is positioned at distal end 202 of shaft 200. In some examples, suture end cap assembly 400 may be fixedly coupled to or integrally formed with distal end 202 of shaft 200. In other examples, suture end cap assembly 400 may be removably coupled to distal end 202. Suture end cap assembly 400 includes a distal face 400a. Distal face 400a may define or include a first working channel opening 480 corresponding to or in communication with working channel 210 of the shaft 200. In an exemplary embodiment, opening 480 may be centrally positioned on distal face 400a. Explained differently, opening 480 may be coaxial or approximately coaxial with axis Z (FIG. 2B) of shaft 200. Distal face 400a may include a first axis, axis X (FIGS. 1B, 2A), and a second axis, axis Y (FIGS. 1B, 2B). Each of axes X, Y may be perpendicular to one another and perpendicular to axis Z. Further, each of axes X, Y to may bisect distal face 400a, for example, with each axis dividing distal face 400a into equal or approximately equal halves. Distal face 400amay further define or include a second working channel opening 490 in communication with or corresponding to second working channel 220 of shaft 200. As will be discussed in further detail below, axis Y may include a center point of each of first working channel opening 480 and second working channel opening 490. In some embodiments, one of axes X, Y may include a center point of each of first working channel opening 480 and second working channel opening 490.

Suture end cap assembly 400 (e.g., distal face 400a) may include various components to facilitate visualization and instrument access during medical procedures. In some aspects, suture end cap assembly 400 may include one or more imaging devices (e.g., cameras, imagers, lens, imaging sensors etc.) to provide visual feedback to the operator. For example, the one or more imaging devices may include an imager 460 positioned on distal face 400a to capture images or video of the target site. Suture end cap assembly 400 may also include one or more lighting elements (LEDs, fiber optic light guides, or other light sources) to enhance visibility of the target or target site during procedures. In the exemplary embodiment, the one or more lighting elements may include two lights 470 positioned on the distal face of distal end 202 illuminate the target site. The arrangement of these components on distal face 400a may be optimized to provide clear visualization while allowing efficient access for instruments through the working channel openings (e.g., working channels openings 480, 490). The specific positioning and configuration of the imager 460 and lights 470 may vary depending on the intended use and design of the medical device.

In some aspects, shaft 200 may include an end cap, different from suture end cap assembly 400, at distal end 202. The end cap of shaft 200 may be fixedly or removably coupled to distal end 202 of shaft 200. The end cap of shaft 200 may include one or more lighting elements (e.g., lights 470) and one or more imaging devices (e.g., imager 460). In some examples, the end cap of shaft 200 may include one or more openings corresponding to one or more of the one or more channels of shaft 200. According to some aspects, the end cap of shaft 200 may include any of the features of suture end cap assembly 400. For example, the end cap of shaft 200 may include another suture assembly 404. In some examples, suturing end cap assembly 400 may be coupled to the end cap of shaft 200. Additionally, suture end cap assembly 400 may not include one or more lighting elements or one or more imaging devices, but instead suture end cap assembly 400 may be coupled to the end cap of shaft 200, with the end cap of shaft 200 including one or more lighting elements and one or more imaging devices. In some aspects, suture end cap assembly 400 may include an end ring, for example, to help couple suture end cap assembly 400 to the end cap of shaft 200.

Still referring to FIGS. 2A-2B, suture end cap assembly 400 includes suture assembly 404. One or more components or elements of suture assembly 404 may be fixedly attached to or integrally formed with suture end cap assembly 400. For example, one or more elements of components may be fixedly attached distal face 400a. Suture assembly 404 may include a needle guard 410, a tissue guard 412, a needle driver 420, and a needle assembly 450.

Needle guard 410 may be fixedly attached to suture end cap assembly 400. For example, needle guard 410 may be fixedly attached to distal face 400a and may extend distally from distal face 400a. Needle guard 410 may include a "U" shape with the legs of the "U" being fixedly attached to distal face 400a. Needle guard 410 may define a slot or space 411 between the legs of the "U" sized and shaped to receive needle driver 420 and needle assembly 450.

Similarly, tissue guard 412 may be fixedly attached to suture end cap assembly 400. In some examples according to the disclosure, tissue guard 412 may be fixedly attached to distal face 400a and may extend distally from distal face 400a. Tissue guard 412 may include a hollow, generally cylindrical shape. Tissue guard 412 may be positioned on distal face 400a to be coaxial or approximately coaxial with second working channel opening 490 and extend distally from opening 490. Tissue guard 412 may include an angled, distal portion. The angled, distal portion may be configured such that a distalmost end of tissue guard 412 is a radially outer to a distal end of tissue guard 412 that is diametrically opposed to the distalmost end of tissue guard 412. Explained differently, the distal portion of tissue guard 412 may be angled proximally in a direction radially inward, for example, toward axis Z. The angled portion of tissue guard 412 may help to provide support for the tissue receiving the suture, for example, helping to allow needle assembly 450 to more easily penetrate the tissue and be received through one or more of second working channel 220, second working channel opening 490, or a lumen of tissue guard 412.

As shown in FIG. 2B, suture assembly 404 includes a needle assembly 450. A suture 448, or a portion thereof, may be coupled to needle assembly 450. Suture 448 may be slidably received within second working channel 220 via port 142. Needle assembly 450 may include a first end 450a and a second end 450b. First end 450a may be removably couplable to needle driver 420. Second end 450b may include a sharp or pointed tip configured for penetrating or piercing tissue. As shown in FIG. 2B, when needle assembly 450 is not coupled to needle driver 420, needle assembly 450 and a portion of suture 448 may be received within one or more of tissue guard 412 (e.g., the lumen of tissue guard 412) or second working channel 220 (e.g., via second working channel opening 490).

Needle driver 420 may include a first arm 422 and a second arm 424. A first end of first arm 422 may pivotally coupled to needle guard 410 at pivot point 414. A second end of first arm 422 may be coupled to a first end of second arm 424. Second arm 424 may extend from the first end of second arm 424 at the second end of first arm 422 to a second end of second arm 424. At least a portion of second arm 424 may include an arcuate curve. The second end of second arm 424 may include a needle coupler 432. Needle coupler 432 may be configured to be releasably couplable to needle assembly 450. For example, needle coupler 432 may be releasably coupled to first end 450a of needle assembly 450.

When viewed from the perspective (proximal facing) of FIG. 2A, suture assembly 404 may be positioned on suture end cap assembly 400 such that axis Y bisects needle coupler 432 and needle assembly 450. In some aspects, axis Y may bisect needle coupler 432 and first working channel opening 480. In some aspects, axis Y may bisect a longitudinal axis of suture assembly 404 and intersect a center point of first working channel opening 480. Explained differently, a plane extending along axis Z and including axis Y may bisect needle coupler 432, needle assembly 450, and first working channel opening 480. The position of first working channel opening 480 and needle coupler 432 may help the operator in aligning tissue with the path that needle assembly 450 follows when needle driver 420 is transitioned to the closed position. For example, end effector 504 may be coupled at a desired suture location on the tissue and then be proximally retracted (e.g., along axis Z) toward first working channel opening 480 until the desired suture location is aligned with the sharp tip of needle assembly 450. In other words, the desired suture location on the tissue may be more easily aligned with the path of needle assembly 450.

Further, the arrangement of needle coupler 432 and opening 480 along axis Y may help to provide intuitive control for an operator when positioning needle assembly 450 relative to the tissue. The operator may use the centrally located first working channel opening 480 as a reference point for aligning the needle assembly 450 with a desired position on the tissue. Using the centrally located first working channel opening 480 as a reference point for aligning needle assembly 450 with a desired position on the tissue may help the operator in consistently delivering full-thickness sutures (e.g., sutures extend through an interior surface of the tissue and an exterior surface of the tissue). This may help to improve accuracy and consistency when placing sutures.

In certain embodiments, when viewed from the viewing angle of FIG. 2A, axis Y may bisect needle coupler 432, first working channel opening 480, and second working channel opening 490. In other words, the plane extending along axis Z and including axis Y may bisect needle coupler 432, needle assembly 450, first working channel opening 480, and second working channel opening 490. In this arrangement, the path that needle assembly 450 follows may be aligned with tissue guard 412 (e.g., the angled opening of tissue guard 412), such that needle assembly 450 coupled to needle coupler 432 is receivable within tissue guard 412 and second working channel opening 490. Axis Y may intersect suture assembly 404 and include center points of openings 480, 490. In other aspects, one or more of a longitudinal axis of needle guard 410, a central longitudinal axis of first working channel opening 480, and a central longitudinal axis of second working channel opening 490 may be intersected by axis Y.

In some aspects, axis X may include the center point of first working channel opening 480, but not include the center point of second working channel opening 490. For example, in some examples, the center point of first working channel opening 480 may be offset from axis X (e.g., non-coaxial with axis Z). In an exemplary embodiment, the center point of first working channel opening 480 may be positioned nearer the center point of second working channel opening 490. In another exemplary embodiment, the center point of first working channel opening 480 may be positioned near to needle guard 410 than the center point of second working channel opening 490. In other words, the center point of first working channel opening 480 may be positioned on one side of axis X and needle guard 410 may be positioned on another, opposite side of axis X.

Referring back to FIG. 1A, medical system 100 may include a needle exchange device 440. Exchange device 440 may include a handle 442 and a shaft 444 extending distally from a distal end of handle 442. Handle 442 may include an actuator 446 at a distal end of handle 442. A distal end of shaft 444 may be inserted through port 140 of insertion device 110 and extended distally though second working channel 220. As shown in FIG. 2B, insertion device 110 may include a needle anchor 452 within a distal portion of second working channel 220. Needle anchor 452 may be configured to releasably couple to a portion of needle assembly 450, for example, when needle assembly 450 is proximally inserted through second working channel opening 490 into working channel 220. Needle exchange device 440 may be actuated via actuator 446 to decouple needle assembly 450 from needle anchor 452. For example, actuating needle exchange device 440 may distally extend the distal end of shaft 444 to decouple needle assembly 450 from needle anchor 452. In some examples, actuating needle exchange device 440 may be configured to couple needle assembly 450 to needle coupler 432.

Needle driver 420 may be actuated to transition needle driver 420 from a first, open position (FIG. 2B) to a second, closed position (not shown). In the open position, first arm 422 may extend radially outward from needle guard 410, and needle coupler 432 may be positioned within slot 411 of needle guard 410. Further, in the open position, a longitudinal axis of first arm 422 may be approximately perpendicular to axis Z. When needle coupler 432 is coupled to needle assembly 450, at least a portion of needle coupler 432 and needle assembly 450 may be positioned within slot 411. In some aspects, when needle coupler 432 is coupled to needle assembly 450, at least a portion of needle assembly 450 (e.g., the sharp tip of second end 450b) may be positioned within slot 411. In the closed position, the longitudinal axis of first arm 422 may be approximately parallel with axis Z, and at least a portion of second arm 424, including needle coupler 432, may be received within one or more of tissue guard 412 or second working channel 220.

Control member 232, extending through control member channel 230, may be operably connected to needle driver 420 at a distal end of control member 232. Control member 232 may include one or more of a wire (e.g., push wire, pull wire, control wire, etc.), a rod, a cable, a string, etc. For example, a proximal portion of control member 232 may include a wire and a distal portion of control member 232 may include a rod. A proximal end of control member 232 may be operably connected to an actuator of medical system 100. For example, the one or more actuators of handle 112 of insertion device 110 may include a third actuator 136 operably connected to the proximal end of control member 232. A first actuation of third actuator 136 may transition needle driver 420 from the open position (FIG. 2A) to the closed positioned. A second actuation, or de-actuation of third actuator 136 may transition needle driver 420 from the closed position to the open position. Actuating third actuator 136 to transition needle driver 420 may provide sufficient force to removably couple needle coupler 432 to needle assembly 450 or to removably couple needle assembly 450 to anchor 452.

Now referring to FIGS. 1A and 2B, medical instrument 500 (e.g., an end effector assembly) may include a handle 506 and a shaft 502 extending distally from a distal end of handle 506 to a distal end of shaft 502. As mentioned, the distal end of shaft 502 may include an end effector 504 configured to couple to tissue at the target site. End effector 504 may include a corkscrew or helical shape (e.g., a helix). A distal end of end effector 504 may include a pointed or sharp tip configured for penetrating or piercing tissue. Although end effector 504 is described as including a helical shape, it should be understood that end effector 504 may be any type of end effector configured to couple to tissue, such as forceps, a clamp, a grasper, etc. Shaft 502 may be distally extended through first working channel 210 via port 142. In some embodiments, a central longitudinal axis of end effector 504 may be coaxial with axis Z.

As discussed above, in some embodiments, medical instrument 500 may be integrated with a medical device (e.g., insertion device 110) of system 100. In an exemplary embodiment, insertion device 110 may include a sheath within first working channel 210. The sheath of insertion device 110 may extend longitudinally through at least a portion of first working channel 210. The sheath of insertion device 110 may be fixedly coupled to insertion device 110. For example, the sheath of insertion device 110 may be fixedly coupled near a distal end of working channel 210 (e.g., first working channel opening 480) or near a proximal end of working channel 210 (e.g., port 142). The sheath may include an inner diameter that is greater than an outer diameter of shaft 502 but lesser than an outer diameter (e.g., width) of end effector 504. Accordingly, shaft 502 may be slidably received within a lumen of the sheath, and end effector 504 may be positioned distal to a distal end of the sheath. Explained differently, the sheath of insertion device 110 may function as a bearing fixedly attached to insertion device 110 and slidably receive shaft 502 therethrough. The inner diameter of the sheath may permit the user to distally extend or proximally retract shaft 502 and end effector 504 relative to distal end 202 of shaft 200. The inner diameter of the sheath may prevent removal of end effector 504 or medical instrument 500 from first working channel 210 due to the outer diameter of end effector 504 being larger than the inner diameter of the sheath. As mentioned, in other aspects, medical instrument 500 may be fully removable or otherwise not integrated with another medical device of system 100 such as insertion device 110.

When needle driver 420 is in the open position (FIG. 2B) and coupled to needle assembly 450, the operator may further extend end effector 504 distally toward the tissue at a target site. The operator may then couple end effector 504 to the tissue, for example, by rotating handle 506, which rotates shaft 502 and end effector 504 in the same direction as handle 506. Once end effector 504 is coupled to the tissue, the operator may proximally retract shaft 502 to pull or otherwise urge the tissue toward distal face 400a of suture end cap assembly 400. In these aspects, the tissue may be aligned with needle assembly 450 (e.g., between needle assembly 450 coupled to needle coupler 432 and tissue guard 412).

In an exemplary procedure, the operator may begin by inserting shaft 200 of insertion device 110 through a natural orifice (e.g., a mouth and an esophagus) of the patient and navigating to a target site to position suture end cap assembly 400 proximate to tissue at the target site. Needle driver 420 may initially be in the open position and with suture assembly 450 coupled to needle anchor 452 (e.g., instead of being coupled to needle coupler 432). The operator may then couple needle assembly 450 to needle coupler 432 by transitioning needle driver 420 to the closed position, such that needle coupler 432 is received within second working channel 220 and coupled to needle assembly 450. Using the exchange device 440, the operator may actuate exchange device 440 to decouple needle assembly 450 from needle anchor 452. After decoupling the needle assembly 450 from needle anchor 452, the operator may transition needle driver 420 from the closed position to the open position.

Now, with needle coupler 432 coupled to needle assembly 450 and needle driver 420 in the open position, the operator may distally extend end effector 504, couple end effector 504 to the tissue, and proximally retract end effector 504 and the coupled tissue, such that the tissue is aligned with the sharp tip of needle assembly 450. The operator may again transition needle driver 420 from the open position to the closed position to pierce the tissue with needle assembly 450. As discussed above, suture 448 may be coupled to needle assembly 450 such that suture 448 may be threaded through the opening formed by needle assembly 450 as needle driver 420 transitions to the closed position. After piercing the tissue, needle assembly 450 may be received with second working channel 220 and re-coupled to needle anchor 452. After which, the operator may again transition the needle driver from the closed position to the open position to decouple needle coupler 432 from needle assembly 450. The operator may reposition or reorient suture end cap assembly 400 by rotating shaft 200 (e.g., by rotating distal portion 112d of handle 112), and repeat the above-described steps to deliver additional sutures to the tissue. Additionally, or alternatively, the operator may reposition distal end 202 of shaft 200, and repeat the above-described steps to deliver additional sutures.

Referring now to FIGS. 2B and 3A-5B, when using medical instrument 500, the operator may control a length of a suture delivered to the tissue coupled to end effector 504 by adjusting a distance D between the distal end of end effector 504 and needle coupler 432 or needle assembly 450 coupled to needle coupler 432. The operator may adjust distance D by proximally retracting or distally extending end effector 504 relative to needle coupler 432. As illustrated in FIGS. 3A-5B, suture assembly 404 may be configured to deliver longer sutures when end effector 504 is closer to distal face 400a and further from needle coupler 432, that is, when distance D is larger. Similarly, suture assembly 404 may be configured to deliver shorter sutures when distance D is shorter.

FIGS. 3A, 4A, and 5A illustrate suture end cap assembly 400, including suture assembly 404, at a target site 20 with end effector 504 coupled to a first side 30 of the tissue. FIGS. 3B, 4B, and 5B correspond to FIGS. 3A, 4A, and 5B respectively. FIGS. 3B, 4B, and 5B depict a second side 40 of the tissue, opposite to first side 30. In other words, first side 30 may correspond to an interior surface of a bodily lumen or cavity and second side 40 may correspond to an exterior surface of the bodily lumen or cavity. An actuation of suture assembly 404 to deliver a suture may form a first opening through sides 30, 40 and a second opening through sides 30, 40. The second opening may be offset from the first opening by a distance corresponding to distance D. The portion of the applied suture extending along second side 40 between the first and second openings may include a length (e.g., lengths L1, L2, L3) corresponding to distance D when the suture is delivered. As mentioned, the arrangement of first working channel opening 480 relative to needle coupler 432 may permit sides 30, 40 of tissue to be retracted proximally (e.g., via end effector 504) to a position between needle assembly 450 coupled to needle coupler 432 and tissue guard 412. Positioning sides 30, 40 of tissue between needle assembly 450 coupled to needle coupler 432 and tissue guard 412 may help the operator in delivering sutures that fully extend a thickness of tissue between first side 30 and second side 40.

For example, as shown in FIG. 3A, distance D between the distal end of end effector 504 and needle assembly 450 is relatively small. Accordingly, a delivered suture 448A in FIG. 3B may include a first length L1 along second side 40 that is relatively short. For example, first length L1 may be approximately 4 mm. As shown in FIG. 4A, distance D between the distal end of end effector 504 and needle assembly 450 is relatively moderate or intermediate. Accordingly, a delivered suture 448B in FIG. 4B may include a second length L2 along second side 40 that is relatively moderate or intermediate in length. For example, second length L2 may be approximately 7 mm. Compared to FIG. 3A, the relative larger distance D in FIG. 4A may result in a larger portion of tissue being positioned proximal to needle coupler 432, which may result in a larger length L2 of suture 448B. Finally, as shown in FIG. 5A, distance D between the distal end of end effector 504 and needle assembly 450 is relatively long. Accordingly, a delivered suture 448C in FIG. 5B may include a third length L3 along second side 40 that is relatively long. For example, the third length L3 may be approximately 10 mm.

Although the disclosed medical devices and medical instruments are described herein as being a component of system 100, it should be understood that one or more of the disclosed medical devices and medical instruments may be used separately from system 100 or integrated into a single medical device.

It will be apparent to those skilled in the art that various modifications and variations can be made to the disclosed medical devices, systems, and related methods without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical system comprising:
a handle;
a shaft extending from a proximal end of the shaft at a distal end of the handle to a distal end of the shaft, the shaft including one or more channels extending from the proximal end of the shaft to a distal opening at the distal end of the shaft, the one or more channels include a first working channel;
a suture end cap assembly extending distally from the distal end of the shaft, the suture end cap assembly including:
a needle,
a needle guard, and
a first working channel opening corresponding to the first working channel;
wherein a common axis intersects a longitudinal axis of the suture end cap assembly and a center point of the first working channel opening; and
an end effector assembly including a shaft and an end effector at a distal end of the shaft of the end effector assembly, the shaft of the end effector assembly is slidably received within the first working channel.

2. The medical system of claim 1, wherein the common axis is a first common axis, wherein a second common axis, perpendicular to the first common axis, includes the center point of the first working channel opening.

3. The medical system of claim 1, wherein the common axis is a first common axis, wherein the center point of the first working channel opening is positioned opposite to the needle guard relative to a second common axis, perpendicular to the first common axis.

4. The medical system of claim 1, wherein the one or more channels includes a second working channel, wherein the suture end cap assembly includes a second working channel opening corresponding to the second working channel.

5. The medical system of claim 4, wherein the common axis intersects a center point of the second working channel opening.

6. The medical system of claim 1, wherein the system further includes a control member, wherein the one or more channels of the shaft includes a control member channel, wherein the control member is slidably positioned within the control member channel.

7. The medical system of claim 6, wherein the common axis intersects a center point of the control member channel.

8. The medical system of claim 7, wherein the control member is actuatable to control a position of the needle.

9. The medical system of claim 1, wherein the handle includes a proximal portion and a distal portion, wherein the distal portion of the handle is rotatable relative to the proximal portion of the handle.

10. The medical system of claim 9, wherein the shaft is coupled to and rotatable with the distal portion of the handle.

11. The medical system of claim 1, wherein the end effector assembly is movable relative to the suture end cap assembly to control a length of a suture delivered by the suture end cap assembly to tissue.

12. The medical system of claim 1, wherein the suture end cap assembly is fixedly coupled to the distal end of the shaft.

13. The medical system of claim 1, wherein the end effector assembly is integrated with the handle.

14. The medical system of claim 1, wherein the distal end of the shaft includes one or more lighting elements and one or more imaging elements.

15. The medical system of claim 1, wherein a central longitudinal axis of the end effector assembly is coaxial with a central longitudinal axis of the shaft.

16. A medical device comprising:
a handle;
a shaft extending from a proximal end of the shaft at a distal end of the handle to a distal end of the shaft, the shaft including a first working channel and a second working channel, each of the first working channel and the second working channel extending from the proximal end of the shaft to a distal opening at the distal end of the shaft;
a suture end cap assembly at the distal end of the shaft, the suture end cap assembly including:
a needle driver,
a needle coupled to the needle driver, and
a needle guard;
wherein a common plane extending along a central longitudinal axis of the shaft bisects the needle and a center point of the first working channel opening.

17. The medical device of claim 16, wherein the needle driver is actuatable to move and intersect an axis perpendicular to the central longitudinal axis.

18. The medical device of claim 16, further comprising an end effector assembly integrated with the handle, the end effector assembly including a shaft, and an end effector at the distal end of the shaft, wherein a portion of the end effector assembly is positioned within the first working channel.

19. The medical device of claim 18, wherein the end effector includes a corkscrew or helical shape.

20. A medical device comprising:
a handle;
a shaft extending from a proximal end of the shaft at a distal end of the handle to a distal end of the shaft;
a suture end cap assembly at the distal end of the shaft, the end cap assembly including:
a distal face,
a suture assembly including a needle and a needle guard, wherein the needle guard includes a longitudinal axis and is connected to the distal face,
a first distal opening in communication with one or more lumens of the shaft, the first distal opening including a central axis,
a second distal opening in communication with the one or more lumens of the shaft, the second distal opening including a central axis;
wherein a common axis intersects the central axis of the first distal opening, the central axis of the second distal opening, and the longitudinal axis of the needle guard.
